(19) United States
(12) Patent Application Publication
(10) Pub. No.: US 2026/0207032 A1
(43) Pub. Date: Jul. 23, 2026

(54) Method for Optical Calibration and Identification

(71) Applicant: KARL STORZ SE & Co. KG, Tuttlingen (DE)

(72) Inventors: Simon Haag, Rietheim-Weilheim (DE); Jasmin Ilg, Dürbheim (DE); Lukas Buschle, Mühlheim-Stetten (DE)

(73) Assignee: KARL STORZ SE & Co. KG, Tuttlingen (DE)

(21) Appl. No.: 19/136,417

(22) PCT Filed: Dec. 4, 2023
(86) PCT No.: PCT/EP2023/084120

§ 371 Date: Jun. 6, 2025

(30) Foreign Application Priority Data
Dec. 7, 2022 (DE) 102022132577.6

(51) Int. Cl. A61B 1/00; A61B 90/00

(52) U.S. Cl. CPC A61B 1/00057; A61B 1/00059; A61B 90/361

(57) ABSTRACT

Method for providing information by marking instruments for use in endoscopic procedures, by means of which, on the one hand, details of the respective instruments can be identified and, on the other hand, calibrations such as spectral calibrations for white balance or geometric calibrations for measurements, can be carried out.

### TECHNICAL FIELD

The present invention relates to a method for providing information by marking instruments, marking elements and tools provided with marking elements for use in such a method.

### BACKGROUND

Endoscopic surgical techniques have become established for a wide range of surgical procedures. In this case, an endoscopic instrument, which may in particular comprise an endoscope and one or more endoscopic instruments, is guided through a natural body opening or an artificial body opening created with the aid of an incision, to a surgical region located inside the body. For this purpose, endoscopic instruments have an elongate shaft, at the distal end, i.e. end remote from the user, of which a tool for performing surgical manipulations is arranged, which can be actuated by a handle arranged at the proximal end of the shaft, i.e. end close to the user, or by connecting motor drives via an elongate transmission element arranged in the shaft. The shaft can be rigid or flexible. During an endoscopic procedure, the handle or connection remains outside the body opening, while the shaft with the tool is inserted through the body opening.

In this case, the endoscopic instruments differ, among other things, in terms of the connected tool and thus in their function. There are also endoscopic instruments in which the tool can be detached from the shaft and replaced by another one. In particular, after an endoscopic procedure, the instrument can be disassembled for cleaning and/or sterilization.

Endoscopic instruments are usually used multiple times and the endoscopic surgical equipment usually includes several units of the respective endoscopic instruments. Methods are known in the prior art, in particular using artificial intelligence, by means of which the instrument type can be recognized. However, these methods do not yet allow for detection and/or classification down to the level of the serial number and/or other detailed characteristics of the instrument.

Endoscopic procedures are usually performed with video support. This means that in addition to the endoscopic instruments, an imaging device is also inserted into the body and corresponding images of the surgical region and the instruments in use are taken and displayed on a suitable playback device. In the presentation, the correct color reproduction is of paramount importance, especially for the correct identification of tissues. In this case, the color representation in the image chain is influenced by various components, such as the light source used, the optics used and/or the camera head. In order to obtain the best possible endoscopic image, it is advisable to perform a white balance before using the endoscopic system, in order to match the various components used to one another. For manual white balance, according to the methods known from the prior art the user points the endoscope at a white or gray surface and the white balance is carried out, for example by pressing a corresponding button, in the control software. Subsequent changes, such as heating of the light source or changes in sensor efficiency due to temperature changes, cannot be compensated for during the procedure, according to the methods known from the prior art, without removing the camera from the actual site of use. Removing an endoscope during surgery to perform calibrations is possible in principle, but is complicated and time-consuming. Accordingly, recalibrations on site are preferred.

In addition, geometric measurements are often carried out during endoscopic procedures, for example to measure structures, such as tumors. In this case, it proves difficult to obtain absolute measurements, not least because of optical falsifications, which can vary depending on the optics used. Distortions, among other things, come into play here.

Depth determination using artificial intelligence algorithms is known in the prior art in the field of endoscopic measurements with a 2D endoscope, for example from “Digging Into Self-Supervised Monocular Depth Estimation”, Godard et. al., 4 Jun. 2018-arXiv: Computer Vision and Pattern Recognition or “The Temporal Opportunist: Self-Supervised Multi-Frame Monocular Depth”, Watson et. al., IEEE Conference on Computer Vision and Pattern Recognition 2021. The resulting models can predict the depth of a tissue with pixel precision, but do not contain absolute depth information. Similar limitations arise from the application of other computer vision techniques, such as “Structure From Motion” techniques, in which 3D information can be obtained by overlapping time-shifted images using parallax; here, too, no absolute depth information can be obtained.

To compensate for distortions and thus for the calibration of geometric measuring systems, images of predefined geometric structures, such as a checkerboard pattern, are taken before the endoscopic system is used-similar to manual white balance-in order to thus determine the deviations and, if necessary, compensate for them in the display of the images.

### SUMMARY OF THE INVENTION

The object of the present invention is to at least partially overcome the disadvantages known in the prior art. The above object is achieved by methods according to the invention according to claims 1 and 6, a marking region according to claim 9, and by an instrument according to claim 15. The corresponding dependent claims relate to preferred embodiments of the invention.

The present invention discloses markings of instruments for use in endoscopic procedures by means of which, on the one hand, details of the respective instruments can be recognized, i.e. they can be identified, and, on the other hand, calibrations, such as spectral calibrations for white balance or geometric calibrations for measurements, can be carried out, and assistance for endoscopic measurements can also be provided.

Accordingly, the present invention discloses a method for optically calibrating an imaging device, in particular an endoscope system, wherein the imaging device comprises an optical system with optics and an image sensor for recording digital images. The method according to the invention comprises detecting a two-dimensional marking region in the field of view of the imaging device, wherein the marking region comprises optoelectronically readable scripts consisting of lines and/or dots of different widths and gaps therebetween, and the marking region has at least one calibration element with calibration information, detecting the calibration element, and calibrating the imaging device based on the calibration information.

An imaging device within the meaning of the present invention can be any technical and/or electronic device which is suitable for recording, further processing and/or forwarding an image of a viewing region and for displaying it, for example on a screen. Medical imaging devices within the meaning of the present invention can be all types of endoscopes known to a person skilled in the art, in particular dual endoscopes and stereo endoscopes. In this case, an endoscope is a usually narrow and elongate imaging device which is suitable for insertion into a cavity or through a usually small opening and for recording an image of a viewing region within the cavity and/or the region behind the small opening by means of at least one camera or at least one image sensor. Furthermore, room cameras can also be imaging devices within the meaning of the present invention.

Two-dimensional in the sense of the present invention refers in particular to marking regions whose information content is based on a two-dimensional representation. This relates in particular to the application of corresponding two-dimensional marking regions on three-dimensional surfaces or surfaces with a three-dimensional structure, such as the surface of an instrument or the round shaft of an instrument.

Marking regions comprising optoelectronically readable scripts within the meaning of the present invention are in particular barcodes, i.e. representations consisting of parallel lines and gaps of different widths and/or so-called QR codes, i.e. matrices consisting of black and white squares. Accordingly, data are represented in the form of binary symbols. These data can be read mechanically using optical reading devices, such as cameras, and further processed electronically. Further processing here includes both the extraction of the information represented directly and indirectly by means of the binary symbols, as well as the use of this information. Directly represented information is information encoded by binary symbols, indirectly represented information is information represented by the geometric shape (e.g. size) or the color design.

Calibration elements within the meaning of the present invention are any representation of information, in particular representations of certain predefined color values or predefined geometric patterns, on the basis of which an optical calibration can be carried out. Accordingly, calibration elements can have a specific color tone, such as pure white, or a predefined gray tone, over which spectral calibrations, such as white balance, are carried out. If the material of the instrument has a known color, an untreated portion can be used as a calibration element. Calibration elements for geometric calibrations are in particular patterns or shapes with known dimensions. In particular, the entire marking region or parts of the marking region can also be provided as a calibration element.

A white balance in the sense of the present invention is a tonal value correction by which a color-neutral image reproduction, i.e. image reproduction without a color cast, can be achieved on a corresponding output device, such as a screen. In this case, tonal value corrections can also be carried out separately for all existing color channels, usually the primary colors red, green and blue, and the corresponding tonal values of the color channels must be calculated to the same brightness for white balance. For the white balance, the brightest point in each color channel can be sought in an image, and the corrections for the individual color channels can be calculated using their tonal values. White balance is problematic if there are no points in the input image that is to be used for white balance that correspond to the white point. Accordingly, gray or white reference areas can be used, although with white reference areas it is important to ensure that the tonal values are not saturated. According to the invention, the reference areas can be provided as calibration elements as part of a marking region arranged on an instrument.

In embodiments of the method according to the invention, the method may further comprise detecting at least two positioning elements, wherein the positioning elements may be part of the marking region, may be integrated in the marking region or may be located in the immediate vicinity of the marking region.

Positioning elements within the meaning of the present invention are optically recognizable structures or information which facilitate the reading of information stored in the marking region and/or define certain regions. In this way, the calibration elements can be defined as such via positioning elements and, optionally, automatically recognized and read out and/or the corresponding calibration routine can be started automatically.

Accordingly, in embodiments of the method according to the invention, the positioning elements can identify the position of the at least one calibration element.

In embodiments of the method according to the invention, the at least one calibration element can be dimensioned such that it can be resolved by the image sensor. The limit value for structures that can be resolved by a sensor can be determined by the Nyquist limit and depends accordingly on the hardware used. In embodiments of the method according to the invention, the calibration information may include information regarding the geometric and/or spectral calibration. In preferred embodiments of spectral calibration, the identification element is dimensioned such that at least one pixel of the sensor is completely filled when detected by the sensor. In preferred embodiments of geometric calibration, the marking element is dimensioned such that the structures can be resolved with high contrast.

In embodiments of the method according to the invention, the calibration element for spectral calibration can be at least partially monochrome and unstructured. The color to be used for calibration is not fixed and can be selected depending on the application, with white and shades of gray, similar to gray cards in classic photography, being particularly preferred. White balance as a specific form of spectral calibration can be adapted to specific applications, such as the use of white light, hyperspectral imaging techniques and multispectral imaging techniques. In hyperspectral imaging or multispectral imaging, spatially and wavelength-resolved detection in different electromagnetic ranges (spectrum) can be used to detect and visualize structures or objects that are not visible using conventional imaging techniques. In preferred embodiments, the calibration element may be unstructured and/or matte to prevent optical interference effects.

The present invention is further directed to a method for identifying instruments, in particular instruments for use in an endoscope system. The method according to the invention comprises detecting a two-dimensional marking region in the field of view of an imaging device, wherein the marking region comprises optoelectronically readable scripts consisting of lines and/or dots of different widths and gaps therebetween, and the marking region encodes at least identification information.

In embodiments of the method for identifying instruments, the imaging device can be a room camera or a camera directed at a specific region, such as a surgical trolley, or a specific object, such as a surgical tray.

Marking regions comprising optoelectronically readable scripts within the meaning of the present invention are in particular barcodes, i.e. representations consisting of parallel lines and gaps of different widths and/or so-called QR codes, i.e. matrices consisting of black and white squares, which can represent information to the person skilled in the art in a standardized manner.

The present invention is further directed to identification regions for optical calibration of an imaging device, in particular an endoscope system, and/or for identification of instruments, in particular instruments for use in an endoscope system. Here, the marking regions are optoelectronically readable scripts consisting of lines and/or dots of different widths and gaps therebetween, and contain at least one calibration element, wherein the calibration element comprises information regarding the geometric and/or spectral calibration and wherein the optoelectronically readable scripts encode identification information. Marking regions are in particular barcodes, i.e. representations consisting of parallel lines and gaps of different widths and/or so-called QR codes, i.e. matrices consisting of black and white squares, which can represent information to a person skilled in the art in a standardized manner.

In embodiments of the two-dimensional marking regions according to the invention, at least two positioning elements can be arranged as part of the marking region, integrated into the marking region or in the direct vicinity of the marking region. The positioning elements can be arranged in such a way that they clearly determine the position of the at least one calibration element and thus facilitate recognition and, in certain embodiments, also enable automatic recognition, in particular computer-based, object recognition.

In embodiments of the two-dimensional marking regions according to the invention, the at least one calibration element can be dimensioned such that it can be resolved by an image sensor as described above.

In embodiments of the two-dimensional marking regions according to the invention, the calibration information may include information regarding the geometric and/or spectral calibration as described above. For calibration elements for spectral calibration, these can be at least partially monochrome and unstructured.

The present invention is further directed to an instrument for use in a method for optical calibration as described above and/or in a method for identification as described above, comprising a two-dimensional identification region as described above. In embodiments of the present invention, the two-dimensional marking region can be arranged on the instrument by means of printing, laser marking, etching, stamping and/or labeling. In this case, in preferred embodiments, in particular the basic color of the material of the instrument can be integrated as a component in the marking region.

### BRIEF DESCRIPTION OF THE FIGURES

The accompanying drawings show embodiments of the invention by way of example and serve to explain the principles of the invention by way of example.

FIG. 1 shows a checkerboard pattern (1a) and representations of various distortions (1b to 1e).

FIGS. 2a and 2b show the sequences of the methods according to the invention.

FIG. 3 shows the application of the methods according to the invention.

FIG. 4 shows instruments with marking according to the invention.

FIG. 5 shows a marking region with positioning elements and calibration elements.

FIG. 6 shows a marking region with a calibration element.

FIG. 7 shows a marking region (a) and schematically a perspective distortion on the instrument (b).

FIG. 8 shows a marking region with geometric calibration element (a) and schematically a perspective distortion on the instrument (b).

### DETAILED DESCRIPTION

The present invention will be described in more detail below with reference to the accompanying drawings which show preferred embodiments. However, the teaching of the invention may be embodied in many different forms and is not to be construed as limited to the embodiments shown herein. It should be noted that these figures are intended to illustrate the general features of the methods used in certain embodiments. However, the illustrations may not accurately depict the structure or features of a particular embodiment. Moreover, like reference signs in the figures designate corresponding parts throughout the different views or embodiments.

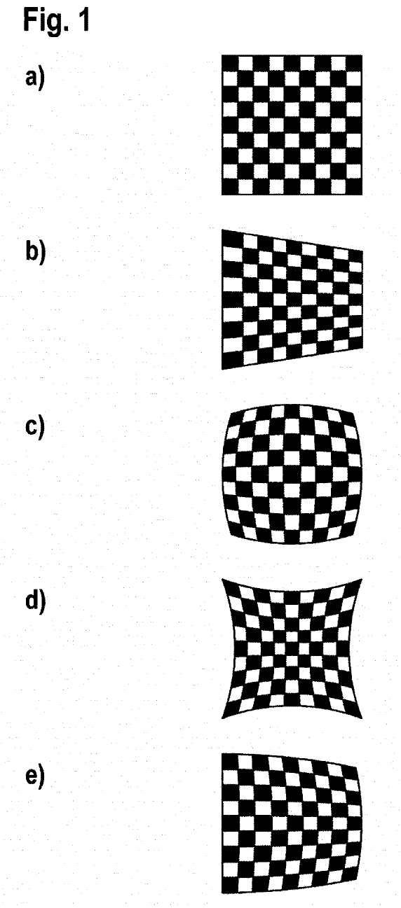

FIG. 1 schematically shows the effects of distortion. Distortion is a rotationally symmetrical image error that increases from the center of the image to the edge of the image. This results in a local change in the image scale within the image plane, which can be very disruptive in measurement applications. If the magnification increases towards the edges of the image field, a square is distorted in a pincushion shape. The opposite case is referred to as barrel distortion. FIG. 1a shows the representation of a checkerboard pattern without distortion. FIG. 1b shows tangential distortion, FIG. 1c shows radial barrel distortion, FIG. 1d shows radial pincushion distortion, and FIG. 1e shows tangential and radial distortion. Which image error or combination of image errors occur(s) depends on the optics used and also on the distance of the object or part of the object shown. By calibrating the system, distortions can be compensated for using digital image processing for the display. While optically-based image errors can usually be compensated for well by a one-time calibration, for example by comparing with a calibration pattern during production, dynamic changes or perspective effects cannot be compensated for in advance. In this case, in particular the possibilities for dynamic changes are so diverse that it is not possible to exclude them in advance, for example through constructive measures. In this case, in particular aging processes come into play, possibly promoted by external parameters such as the autoclaving of the imaging device or external forces during use. In particular, the position of individual components of the optical system relative to one another can change over time, which can have serious effects on image reproduction. Likewise, forces occurring during movements of the imaging device can influence the optics or parts of the optics, such as the relative position of sensors or lenses. In addition, a change in the optics used, such as in an endoscope with a proximal camera head, can also lead to changes; these optics changes cannot be detected by the camera without additional measures. Accordingly, dynamic, preferably automated, calibrations according to the invention by carrying out the calibration routines during use are advantageous here.

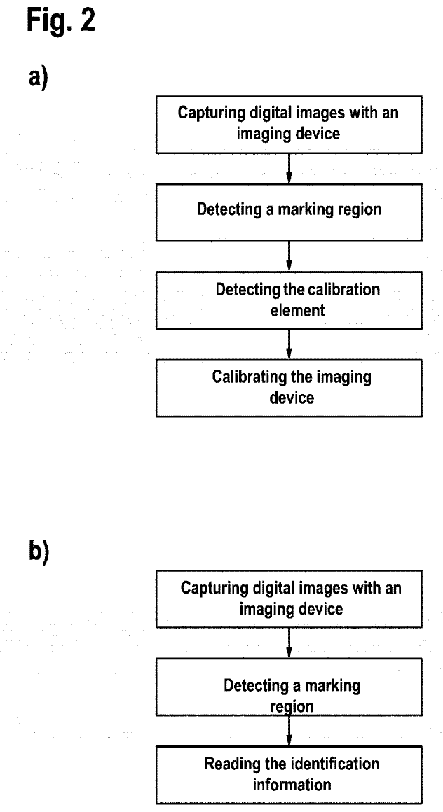

FIG. 2a shows the method for optical calibration of an imaging device, in particular an endoscope system, wherein the imaging device has an optical system with optics and an image sensor for recording digital images. At the beginning of the method, at least one digital image is recorded by means of the imaging device, wherein the recording of digital images can also take place continuously, for example in the form of video streams. When a corresponding marking region comes into the field of view of the imaging device, it is captured together with the calibration element contained therein. The recording can be carried out in particular with the help of appropriate object recognition techniques. The subsequent calibration can then be performed manually by the user using the information contained in the calibration element, semi-automatically, for example by automatic recognition and/or selection of the respective calibration routines, but after initiation by suitable user inputs by the user, or fully automatically. Distortions can be compensated using geometric calibration information, as described in FIG. 1. Since the geometric calibration information is known, distortions caused by the optics used can be determined and compensated for by software for display on the output device. Likewise, spectral calibration information can be used for tonal correction, especially for white balance. For this purpose, a corresponding reference field with known properties, in particular a white or gray reference field, is captured and recognized; appropriate object recognition techniques can be applied here.

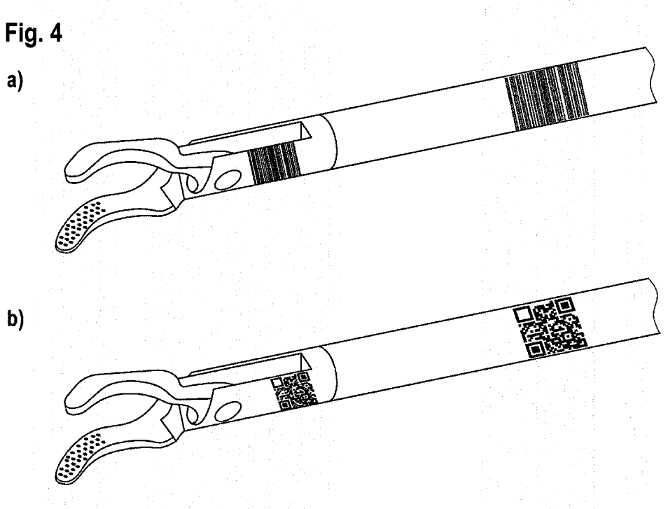

FIG. 2b shows the method for identifying instruments, in particular instruments for use in an endoscope system. At the beginning of the method, at least one digital image is recorded by means of the imaging device, wherein the recording of digital images can also take place continuously, for example in the form of video streams. When a corresponding marking region comes into the field of view of the imaging device, it and the identification information it contains are captured. The recording can be carried out in particular with the help of appropriate object recognition techniques. Based on the identification information, for example the use of the instrument can be logged, instrument-specific settings can be made, or instrument-specific information can be displayed to the user on a display device, such as a monitor. Alternatively or in addition, subsequent instrument-specific calibration routines can be initiated or suggested to the user. Room cameras can also be used as imaging devices for the identification method, as described below in connection with FIG. 4. In addition to documenting the use of the identified instrument, identification also allows for verification, e.g. via certificates of authenticity, or checking suitability for the intended use, and the like. In this way, a user of the system can be given further information via the instrument identification via a corresponding display device. Accordingly, warnings can be displayed if unverified and/or incompatible devices are detected.

In preferred embodiments, the calibration and identification methods are used in combination, i.e. the marking regions contain both calibration elements and identification information.

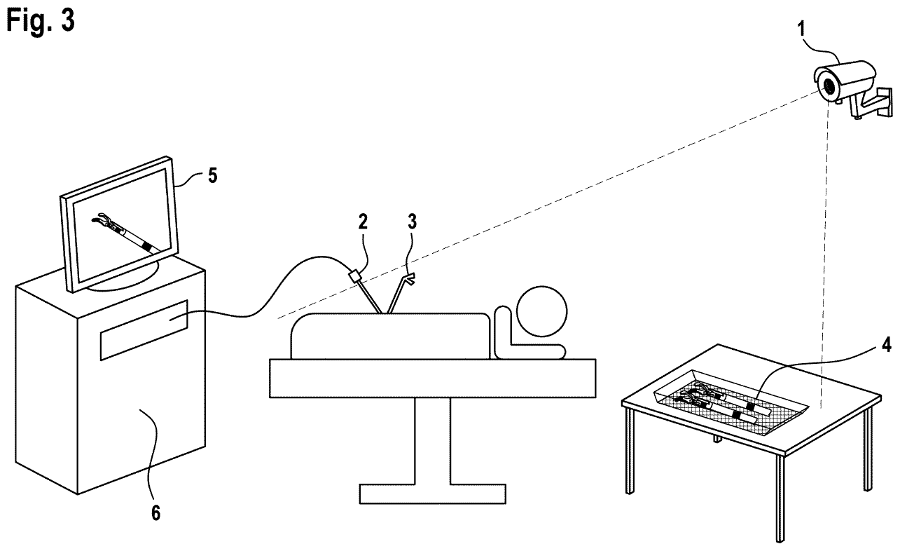

FIG. 3 is a schematic representation of the application of the methods described in FIGS. 2a and 2b in the context of an endoscopic surgery in an operating room. The operating room is equipped with typical endoscopic surgical equipment, including an endoscope with camera 2 and instruments, with one instrument 3 inserted into the endoscope and the remaining instruments in a tray 4. The operating room also has a room camera 1, a control unit 6 and a monitor 5. The instruments located in the sieve 4, as well as the instrument(s) 3 in use, can be identified using the method according to the invention via the room camera 1. In addition, the instruments in use can be identified via the endoscope camera according to the method according to the invention or corresponding calibration routines can be carried out. As described above, the calibration routines can be performed manually, semi-automatically or fully automatically. The digital image of the instrument with the marking region can also be displayed on the monitor 5. Continuous, automatic calibration is also possible. In addition, for example based on digital image analysis, in particular with the aid of artificial intelligence, occurring image changes can be detected and subsequent calibrations can be suggested or carried out. Depending on the calibration, the display on the monitor was done with corrected tonal values and/or compensation of distortions. By regularly detecting the marking regions, any newly occurring deviations, for example due to temperature changes, can be dynamically recorded and, optionally, new calibrations can be carried out or suggested via the monitor 5 as a corresponding note for the user of the system.

As described above, by applying marking regions to an endoscopic instrument 3, it is possible to read this marking region via the endoscopic image captured by the endoscope camera 2. This makes it possible, among other things, to clearly identify and record the instrument type using the serial number encoded in the marking region. By correlating with events (such as the occurrence of bleeding) that occurred with a particular instrument, further analyses can be performed that can lead to an improvement in the indication. By capturing the marking region(s) via the room camera 1, digital sieve inspection can be enabled and thus optimization processes can be carried out.

FIG. 4a shows a marking region in the form of a peripheral barcode on an endoscopic instrument. FIG. 4b shows the labeling of an endoscopic instrument using so-called QR codes. In both cases, distances between individual elements of the marking region or the overall size (length, width, or a combination of length and width) can be used as geometric calibration information before measurements. As described in connection with FIG. 1, calibration steps may be necessary in this case to compensate for image errors. However, since the dimensions of the marking regions are known, these can be used for the respective calibrations, or even the need for calibration can be automatically detected and optionally carried out based on analysis of the digital images.

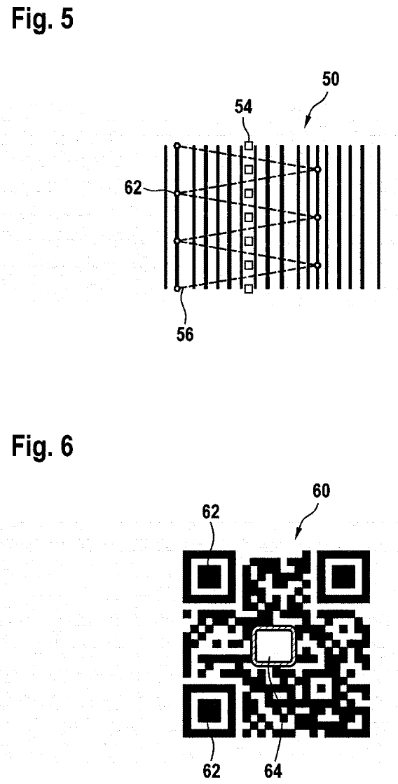

FIG. 5 shows a marking region 50 in the form of a further developed barcode. In addition to the known parallel lines of different thickness, the marking region 50 has calibration elements 54 and positioning elements 52. A calibration element can be defined by triangulation, represented by the auxiliary lines 56. This allows calibration elements 54 to be verified. For example, it can thus be ensured that only calibration elements 54 in suitable alignment with the imaging device (e.g. when three positioning elements 52 are visible) are used for calibrations. The dimensions of the marking region 50 and/or the thickness and spacing of individual lines can therefore be used here as scale or geometric calibration information.

FIG. 6 shows a marking region 60 in the form of a further developed QR code. Here, a calibration element 64 is arranged centrally as a gray portion for spectral calibration, such as for a white balance. The typical corner elements of the QR code serve here as positioning elements 62. The dimensions of the marking region 60 and/or the size of individual squares can therefore be used as scale or geometric calibration information. In this case, the geometric arrangement of the calibration element 64 in the marking region 60 is basically free, but in preferred embodiments it is uniform, the calibration element 64 being arranged at corresponding positions within different marking regions, which can facilitate automated detection.

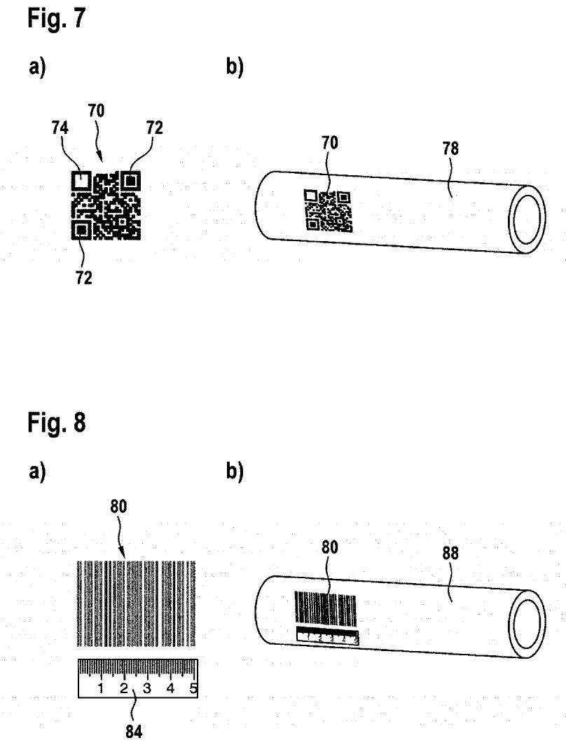

FIG. 7a shows another embodiment of a marking region 70 in the form of a further developed QR code. Here, the calibration element 74 for spectral calibration is arranged as a white field for white balance in one of the corner elements typical for the QR code. The other two corner elements 72 serve as positioning elements. As already described above, the geometric arrangement of the calibration element 74 in the marking region 70 is also free here, but in preferred embodiments it is uniform.

FIG. 7b shows the marking region 70 of FIG. 7a arranged on a portion 78 of an endoscopic instrument, the perspective distortion being illustrated. Knowing the dimensions of the marking region 70, means that the endoscopic system can be calibrated so that absolute measurements are possible. For this purpose, a white balance can be carried out via the calibration element 74—even during use without removing the endoscope from the work region.

FIG. 8a shows another embodiment of a marking region 80 in the form of a further developed barcode. Here the barcode is extended by a geometric calibration element 84.

FIG. 8b shows the marking region 80 of FIG. 8a arranged on a portion 80 of an endoscopic instrument, the perspective distortion being illustrated. Here, the calibration element 84 can be used directly as a scale or also for geometric calibrations.

The scope of this disclosure includes all changes, substitutions, variations, alterations, and modifications to the embodiments described or illustrated herein that would be understood by a person of ordinary skill in the art. The scope of this disclosure is not limited to the embodiments described or illustrated herein. In addition, although this disclosure describes and illustrates respective embodiments herein as encompassing particular components, elements, features, functions, operations, or steps, any of these embodiments may also include any combinations or permutations of any components, elements, features, functions, operations, or steps described or illustrated anywhere herein that a person of ordinary skill in the art would understand. A reference in the appended claims to a method or apparatus or component of an apparatus or system being adapted, arranged, capable, configured, enabled, operable or ready to perform a particular function further includes that apparatus, system or component, regardless of whether it or that particular function is activated, turned on or enabled, as long as that apparatus, system or component is adapted, arranged, capable, configured, enabled, operable or ready to perform it. In addition, although this disclosure describes or illustrates certain embodiments as providing certain advantages, certain embodiments may also provide none, some, or all of these advantages.

1-16. (canceled)

17. A method for optically calibrating an imaging device, in particular an endoscope system, wherein the imaging device has an optical system with optics and an image sensor for recording digital images, comprising:
detecting a two-dimensional marking region in the field of view of the imaging device, wherein the marking region comprises optoelectronically readable scripts comprising at least one of lines or dots of different widths and gaps therebetween, and the marking region has at least one calibration element with calibration information;
detecting the calibration element; and
calibrating the imaging device based on the calibration information.

18. The method of claim 17, further comprising detecting at least two positioning elements, wherein the positioning elements are part of the marking region, are integrated in the marking region, or are located in direct proximity to the marking region.

19. The method of claim 18, wherein the positioning elements indicate the position of the at least one calibration element.

20. The method of claim 17, wherein the at least one calibration element is dimensioned such that it can be resolved by the image sensor.

21. The method of claim 17, wherein the calibration information comprises information regarding at least one of geometric or spectral calibration.

22. The method of claim 21, wherein the calibration element for spectral calibration is at least partially monochrome and unstructured.

23. The method of claim 17, wherein the calibration is carried out automatically upon detection of the at least one calibration element.

24. A method for identifying instruments, comprising:
detecting a two-dimensional marking region in the field of view of an imaging device, wherein the marking region comprises optoelectronically readable scripts comprising at least one of lines or dots of different widths and gaps therebetween, and the marking region encodes at least identification information.

25. The method of claim 24, wherein the imaging device comprises a room camera.

26. The method of claim 24, wherein marking region comprises a surface of a medical instrument.

27. The method of claim 26, wherein the medical instrument comprises an endoscope.

28. The method of claim 26, wherein the two-dimensional marking region further comprises at least two positioning elements arranged as part of the marking region and integrated into the marking region or proximate to the marking region.

29. The method of claim 28, wherein the positioning elements indicate the position of the at least one calibration element.

30. A device, comprising:
a two-dimensional marking region for the optical calibration of an imaging device for the identification of instruments, further comprising:
optoelectronically readable scripts comprising at least one of lines or dots of different widths and gaps therebetween;
at least one calibration element, wherein the calibration element comprises at least one of geometric or spectral calibration information; and
wherein the optoelectronically readable scripts encode identification information.

31. The device of claim 30, wherein the two-dimensional marking region further comprises at least two positioning elements arranged as part of the marking region and integrated into the marking region or in the direct vicinity of the marking region.

32. The device of claim 31, wherein the positioning elements indicate the position of the at least one calibration element.

33. The device of claim 30, wherein the at least one two-dimensional marking region is dimensioned for resolution by an image sensor.

34. The device of claim 30, wherein the two-dimensional marking region further comprises calibration information, the calibration information further comprising at least one of geometric or spectral calibration information.

35. The device of claim 30, wherein the at least one of geometric or spectral calibration information is at least partially monochrome and unstructured.

36. The device of claim 30, wherein the two-dimensional marking region comprises at least one of printing, laser marking, etching, punching, or labeling.
